(19) United States
(12) Patent Application Publication
(10) Pub. No.: US 2011/0059144 A1
(43) Pub. Date: Mar. 10, 2011

(54) ENCAPSULATED HYDROPHOBIC ACTIVES VIA INTERFACIAL POLYMERIZATION

(76) Inventors: Robert B. Fletcher, Midland, MI (US); David L. Malotky, Midland, MI (US); Xiaodong Zhang, Livingston, NJ (US)

(21) Appl. No.: 12/812,583

(22) PCT Filed: Jan. 13, 2009
(86) PCT No.: PCT/US09/30836

§ 371 Date: Sep. 28, 2010

Related U.S. Application Data
(60) Provisional application No. 61/021,364, filed on Jan. 16, 2008.

(51) Int. Cl. A61K 8/02; A61Q 17/04; A61K 8/37; A61K 47/30; A61Q 19/00; A61Q 13/00

(52) U.S. Cl. 424/401; 424/59; 514/543; 514/772.3; 512/4

(57) ABSTRACT

The present invention provides personal care compositions, as well as methods for protecting low viscosity hydrophobic liquid actives.

### FIELD

The present invention relates to personal care compositions.

### BACKGROUND

Many personal care compositions contain water insoluble or hydrophobic actives. Such actives may render the composition greasy feeling. Since aesthetic perception is a key sales driver in the personal care market, this is undesirable. Moreover, there are health concerns associated with unfavorable aesthetic perception of sunscreens or other beneficial personal care compositions, for example, if the feel of the product causes underutilization.

In addition to irritancy and aesthetic difficulties, sunscreen formulators have also previously been challenged by the fact that avobenzone is photolabile. One strategy to overcome such difficulties is to insulate or encapsulate the hydrophobic active. In the past, encapsulation has required high shear and/or solvent exchange processes. Thus, what is needed are improved methods and compositions containing encapsulated or otherwise protected hydrophobic personal care actives.

### SUMMARY

In one embodiment, the present invention provides methods of improving the aesthetics of a personal care composition which contains a hydrophobic active, comprising combining the hydrophobic active with a surfactant and a first interfacially polymerizable component to form an oil phase, and then reacting the first interfacially polymerizable component with a complementary interfacially polymerizable component, thereby encapsulating the hydrophobic active in a polymer shell.

In one embodiment, the present invention provides methods of improving the aesthetics of a personal care composition which contains a hydrophobic active, comprising combining the hydrophobic active with a surfactant and an isocyanate to form an oil phase, and then reacting the isocyanate with an amine, thereby encapsulating the hydrophobic active in a polyurea shell.

In another embodiment, the present invention provides methods of improving the aesthetics of a personal care composition which contains a hydrophobic active, comprising combining the hydrophobic active with a surfactant and an isocyanate to form an oil phase, emulsifying the oil phase with water, combining a multifunctional amine with water, and reacting the isocyanate with the amine, thereby encapsulating the hydrophobic active in a polyurea shell.

In yet another embodiment, the present invention provides methods of improving ease of spread, improving adsorption onto skin, reducing tackiness, and reducing greasiness of a personal care composition which contains a hydrophobic active, comprising combining the hydrophobic active with a surfactant and an isocyanate to form an oil phase, emulsifying the oil phase with water, combining a multifunctional amine with water, and reacting the isocyanate with the amine by adding the amine and water mixture to the emulsion incrementally with mixing, thereby encapsulating the hydrophobic active in a polyurea shell.

### DETAILED DESCRIPTION

In one embodiment, the present invention provides methods of improving the aesthetics of a personal care composition which contains a hydrophobic active, comprising combining the hydrophobic active with a surfactant and a first interfacially polymerizable component to form an oil phase, and then reacting the first interfacially polymerizable component with a complementary interfacially polymerizable component, thereby encapsulating the hydrophobic active in a polymer shell.

Preferred interfacially polymerizable components include diamine and diacid chloride (to form polyamide), diamine and di/polysulfonyl chlorides (to form polysulfonamide), di/polyol and polyacid chlorides or dicarboxylic acid (to form polyester), di/polyol and polychloroformates or phosgene (to form polycarbonate), isocyanate and diol (to form polyurethane), or diamine and isocyanate (to form polyurea). Particularly preferred interfacially polymerizable components include diamine and isocyanate.

Thus, in one embodiment, the present invention provides methods of improving the aesthetics of a personal care composition which contains a hydrophobic active, comprising combining the hydrophobic active with a surfactant and an isocyanate to form an oil phase, and then reacting the isocyanate with an amine, thereby encapsulating the hydrophobic active in a polyurea shell.

“Aesthetics” refers to sensory perceptions, such as appearance, scent, and tactile properties. In one embodiment, the personal care compositions produced by the methods of the present invention demonstrate improved ease of spread and adsorption onto skin. In one embodiment, the personal care compositions produced by the methods of the present invention demonstrate reduced tackiness and greasiness. In one embodiment, the personal care compositions produced by the methods of the present invention demonstrate improved ease of spread, improved adsorption onto skin, reduced tackiness, and reduced greasiness.

“Personal care” relates to compositions to be topically applied to a person (including mouth, ear, and nasal cavities, but not ingested). Examples of personal care compositions include skin care products (e.g., facial cream, moisturizers, leave on and rinse off lotions, sunscreens, foundation, mascara, eye-liner, lipstick, and the like), oral care products (such as toothpastes and rinses), nail care products (such as polish and conditioners), and hair care products (including leave on and rinse off conditioners, styling gels and hairsprays).

“Hydrophobic,” for purposes of this disclosure, refers to a component that is more soluble in dodecane than in water. Such components generally have a log octanol/water partition coefficient greater than 1. Examples may be found in the CRC Handbook of Chemistry & Physics, edited by D. R. Linde, CRC Press, Florida, 74th Ed. (1993-94), Sec. 16, page 24 et seq.

For purposes of this specification, “actives” for personal care refers to any component that imparts a primary personal care benefit to a user, as opposed to solely facilitating creation of the formulation itself. Thus, for example, water is not an active. Examples of personal care actives include typical actives for skin care products (e.g., facial cream, moisturizers, leave on and rinse off lotions, sunscreens, foundation, mascara, eye-liner, lipstick, and the like), oral care products (such as toothpastes and rinses), nail care products (such as polish and conditioners), and hair care products (including leave on and rinse off conditioners, styling gels and hairsprays). The actives should be cosmetically acceptable. “Cosmetically acceptable” refers to ingredients typically used in personal care compositions, and is intended to underscore that materials that are toxic when present in the amounts typically found in personal care compositions are not contemplated as part of the present invention.

In one embodiment, the hydrophobic active is a moisturizer, emollient, sunscreen, conditioner, vitamin, dye, flavor, or fragrance.

In a preferred embodiment, the hydrophobic active is a sunscreen. Examples of sunscreens include paraminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide and zinc oxide, diethanolamine methoxycinnamate, digalloy trioleate, ethyl dihydroxypropyl PABA, glyceryl aminobenzoate, lawsone with dihydroxy acetone, and red petrolatum. Preferably, the hydrophobic active is octyl methoxycinnamate.

Other examples of actives include antibacterial compounds (e.g. triclosan) in toothpaste, polyphenols, flavinoids and isoflavinoids, coenzyme Q10 and derivatives thereof, carotene and derivatives thereof, salicylic acid and derivatives thereof, dehydroepiandrosterone (DHEA), hydrophobic polysaccharides, proteins, including enzymes and peptides, and botanicals.

Vitamins include Vitamin A and esters thereof, Vitamin D and derivatives thereof, Vitamins B3 and B5 and derivatives thereof, Vitamin E and esters thereof, Vitamin F and derivatives thereof, and Vitamin K.

Dyes include liposoluble dyes, such as Sudan red, DC Red 17, DC Green 6, β-carotene, soybean oil, Sudan brown, DC Yellow 11, DC Violet 2, DC Orange 5, and quinoline yellow.

Flavors include flavor oils, such as peppermint, wintergreen, citrus, fruit, vanilla, and cinnamon. Most flavors are hydrophobic, and thus contemplated.

Fragrances include any component which provides a pleasant scent. Examples include scents that are floral, ambery, woody, leather, chypre, fougère, musk, vanilla, fruit, and/or citrus. Fragrances are often oils obtained by extraction of natural substances or synthetically produced. In one embodiment, the fragrance is one of the essential oils.

In one embodiment, the isocyanate is at least one of diphenylmethane diisocyanate (MDI), Polymeric MDI, polymethylene polyphenyl isocyanate (PAPI), toluene diisocyanate (TDI), isophorone diisocyanate (IPDI), 1,4-phenylene diisocyanate, or hexamethylene diisocyanate (HDI). In a preferred embodiment, the isocyanate is PAPI 27 polymethylene polyphenyl isocyanate that contains MDI, available from The Dow Chemical Company.

In one embodiment, the surfactant is an anionic, nonionic, or cationic surfactant, provided that it does not react with isocyanate. In one embodiment, the surfactant is a mixture of surfactants. In one embodiment, the surfactant is a nonionic surfactant, preferably polyoxyethylene lauryl ether.

In one embodiment, the method further comprises emulsifying the oil phase. In one embodiment, the method further comprises emulsifying the oil phase with water. In one embodiment, the method further comprises emulsifying the oil phase with water and a preservative. The oil phase is emulsified under shear.

It should be understood that in embodiments where water is present, the amine can result from conversion of some isocyanate to amine upon contact with water.

Alternatively, in a preferred embodiment, the amine is a separately added component. In one embodiment, the amine is first combined with water. Thus, in one embodiment, the step of reacting the isocyanate with an amine includes first combining an amine with water and then adding this mixture to the emulsion incrementally with mixing.

In one embodiment, the amine is a multifunctional amine. Preferably, the amine is ethylene diamine.

The amount of isocyanate depends upon the desired encapsulated particle size and shell thickness, which can be determined from the following formulae:

$$\begin{matrix}{V}_{T} □ {V}_{S} □ {V}_{C} & Equation 1 \\ \frac{4}{3} □ {( \frac{{D}_{mean}}{2} )}^{3} □ {V}_{S} □ \frac{4}{3} □ {( \frac{{D}_{mean}}{2} □ t )}^{3} & Equation 2\end{matrix}$$

where:

V_{T} is the total particle volume;

V_{S} is the volume attributable to the shell (volume of isocyanate plus a stoichiometric amount of amine (if the amine is a separately added component));

V_{C} is the core volume attributable to the oil phase;

Dₘₑₐₙ is volume average particle size (measured by light scattering); and

t is the shell thickness.

Preferably, for particles less than 4 μm, a shell thickness of greater than 10 nm is required. Preferably, for particles greater than 10 μm, a shell thickness of greater than 100 nm is required.

In another embodiment, the present invention provides methods of improving the aesthetics of a personal care composition which contains a hydrophobic active, comprising combining the hydrophobic active with a surfactant and an isocyanate to form an oil phase, emulsifying the oil phase with water, combining a multifunctional amine with water, and reacting the isocyanate with the amine, thereby encapsulating the hydrophobic active in a polyurea shell.

In yet another embodiment, the present invention provides methods of improving ease of spread, improving adsorption onto skin, reducing tackiness, and reducing greasiness of a personal care composition which contains a hydrophobic active, comprising combining the hydrophobic active with a surfactant and an isocyanate to form an oil phase, emulsifying the oil phase with water, combining a multifunctional amine with water, and reacting the isocyanate with the amine by adding the amine and water mixture to the emulsion incrementally with mixing, thereby encapsulating the hydrophobic active in a polyurea shell.

Other optional ingredients for personal care compositions of the present invention include cosmetically acceptable emollients, sunscreens, surfactants, emulsifiers, preservatives, rheology modifiers, colorants, preservatives, pH adjustors, propellants, reducing agents, fragrances, foaming agents, tanning agents, depilatory agents, flavors, astringents, antiseptics, deodorants, antiperspirants, insect repellants, bleaches, lighteners, anti-dandruff agents, adhesives, polishes, strengtheners, fillers, barrier materials, or biocides.

The moisturizers include 2-pyrrolidone-5-carboxylic acid and its salts and esters, alkyl glucose alkoxylates or their esters, fatty alcohols, fatty esters, glycols and, in particular, methyl glucose ethoxylates or propoxylates and their stearate esters, isopropyl myristate, lanolin or cetyl alcohols, aloe, silicones, propylene glycol, glycerol and sorbitol.

Conditioners include stearalkonium chloride, dicetyldimonium chloride, lauryl methyl gluceth-10 hydroxypropyldimonium chloride, and conditioning polymers such as polyquaternium-10, polyquaternium-24 and chitosan and derivatives thereof.

Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit Nobel), synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, and the oil of cereal germs.

Other suitable emollients include dicaprylyl ether, C₁₂₋₁₅ alkyl benzoate, DC 200 FLUID 350 silicone fluid (from Dow Corning Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

In some embodiments, the personal care composition further comprises an optional rheology modifier as a thickener. Examples of thickeners include polymers, for example, modified or unmodified carboxyvinyl polymers, such as the products sold under the names CARBOPOL and PEMULEN (INCI name: Acrylates/C₁₀₋₃₀ alkyl acrylate crosspolymer; available from Noveon), polyacrylates and polymethacrylates, such as the products sold under the names LUBRAJEL and NORGEL (from Guardian) or HISPAGEL (from Hispano Chimica), polyacrylamides, 2-acrylamido-2-methylpropanesulfonic acid polymers and polymers, which are optionally crosslinked and/or neutralized, for instance the poly(2-acrylamido-2-methylpropane-sulfonic acid) sold by Clariant (INCI name: ammonium polyacryldimethyltauramide), emulsified crosslinked anionic polymers of acrylamide and AMPS, such as those sold under the name SEPIGEL 305 (INCI name: Polyacrylamide/C13-14 Isoparaffin/Laureth-7; from Seppic) and under the name SIMULGEL 600 (INCI name: Acrylamide/Sodium acryloyldimethyltaurate polymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose, associative polymers, for instance associative polyurethanes, polymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (from Hüls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (from Röhm & Haas).

Colorants include pigments, which are used especially in make-up, including metal oxide pigments, titanium dioxide, optionally surface-treated, zirconium oxide or cerium oxide, zinc oxide, iron oxide (black, yellow or red), chromium oxide, manganese violet, ultramarine blue, chromium hydrate and ferric blue, carbon black, pigments of barium, strontium, calcium or aluminum (for example D&C or FD&C), cochineal carmine, mica coated with titanium or with bismuth oxychloride, titanium mica with iron oxides, titanium mica with, especially, ferric blue or chromium oxide, titanium mica with an organic pigment, nacreous pigments based on bismuth oxychloride, goniochromatic pigments, for example pigments with a multilayer interference structure, reflective pigments, for example particles with a silver-coated glass substrate, glass substrate coated with nickel/chromium/molybdenum alloy, glass substrate coated with brown iron oxide, particles comprising a stack of at least two polymer layers, for instance MIRROR GLITTER (from 3M).

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes from the list above may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors, include inorganic and organic acids and bases and in particular aqueous ammonia, citric acid, phosphoric acid, acetic acid, and sodium hydroxide.

Reducing agents include ammonium thioglycolate, hydroquinone and sodium thioglycolate.

Fragrances may be aldehydes, ketones, or oils obtained by extraction of natural substances or synthetically produced as described above. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

### EXAMPLES

The following examples are for illustrative purposes only and are not intended to limit the scope of the present invention. All percentages are by weight unless otherwise specified.

### Example 1

Exemplary encapsulated hydrophobic actives of the present invention contain the components recited in TABLE 1.

TABLE 1

| | | Batch 1 |
|---|---|---|
| | | Wt. % |
| | | (composition) |
| A | Octyl methoxycinnamate (OMC) | 20.00 |
| | PAPI 27 polymethylene polyphenyl isocyanate | 0.66 |
| | that contains MDI | |
| B | CELVOL 205 polyvinyl alcohol (PVA)-5% soln | 20.67 |
| C | Ethylenediamine (EDA)-10% soln | 1.59 |
| D | VEEGUM magnesium aluminum silicate-5% soln | 4.49 |
| E | KELZAN S xanthum gum-1.5% soln | 1.65 |
| F | Water | 50.94 |

The A components are mixed. The B component is emulsified, for example, using a Silverson Model L4RTA with a 2¼ inch high shear emulsification head. A is slowly added to B while mixing, for example, at 1000-1500 rpm. The mixing speed is then increased until the droplet size is about 0.5 microns less than desired final particle size, this speed hereinafter referred to as “the desired-droplet-rate.” Then, C is added dropwise at a mixing speed 1000-2000 rpm below the desired-droplet-rate (“the C-mixing-rate”). The mixing speed is then lowered again to a relatively slow speed, and the mixture is stirred for 3-4 minutes, after which the mixing speed is increased back to the C-mixing-rate, and D, E, and F are added. Afterwards, the mixture is stirred for 3-4 minutes, this composition comprising OMC particles encapsulated with an interfacially polymerized shell.

### Example 2

An exemplary sunscreen base contains the components recited in TABLE 2.

TABLE 2

| | | Batch 2 |
|---|---|---|
| | Component | Wt. % (composition) |
| A | Homosalate | 27.69 |
| | Avobenzone | 16.62 |
| | CORAPAN TQ Diethylhexyl 2,6 Naphthalate | 27.69 |
| | BRIJ 30 polyoxyethylene lauryl ether | 2.80 |
| | BRIJ 35 polyoxyethylene lauryl ether | 2.80 |
| | EMERSOL 315 linoleic acid | 1.00 |
| | GANEX V216 Alkylated Polyvinylpyrrolidone | 0.80 |
| | Phenoxyethanol | 0.44 |
| | Sorbic Acid | 0.08 |
| | Benzoic Acid | 0.08 |
| B | DI Water | 19.76 |
| | EDTA | 0.04 |
| | Phenoxyethanol | 0.12 |
| | Sorbic Acid | 0.04 |
| | Benzoic Acid | 0.04 |

A is combined and placed in an oven at about 80° C. until the solid ingredients have dissolved. Then, A is removed from the oven and cooled to below about 40° C. B is combined, and then A and B are combined, preferably homogenized, such as by using an IKA mixer.

### Example 3

Formulations made substantially according to the protocols described above in Examples 1 and 2 were made and combined as recited in Table 3, along with a comparative component, to afford sunscreen compositions:

TABLE 3

| | Batch 3 | |
|---|---|---|
| | (Comparative) | Batch 4 |
| Batch 1 | — | 31% |
| DDS 621 emulsified OMC | 9.55% | — |
| Batch 2 | 18.05% | 18.05% |
| CARBOPOL 940 carboxyvinyl polymer | 0.2% | 0.2% |
| Water | 72.2% | 50.75% |

The order of addition was base sunscreen (Batch 2) mixed with either Batch 1 or DDS 621 emulsified OMC (The Dow Chemical Company) and CARBOPOL polymer, then water.

Ten panelists applied 0.05 g of each sample to a designated area on their right or left forearms. Initially, each sample was evaluated for ease of spread, adsorption onto skin, slip, tackiness, greasiness, moist feel, and overall skin feel, and then again after one hour for moist feel, greasiness, skin smoothness, and skin softness, and overall skin feel. The evaluation scale was 0-10, with 10 being the best. For these criteria, the sample according to the present invention (Batch 4) performed as well or better than the comparative sample (Batch 3) with the exception of initial moist feel, as shown in TABLE 4.

TABLE 4

| | | Batch 3 | |
|---|---|---|---|
| | Batch 4 | (Comparative) | Difference |
| Initial ease of spread | 6.2 | 4.8 | 1.4 |
| Initial adsorption | 7.3 | 4.8 | 2.5 |
| Initial slip | 5.4 | 5.4 | 0 |
| Initial tackiness | 8 | 4.6 | 3.4 |
| Initial greasiness/stickiness | 7.2 | 5.6 | 1.6 |
| Initial moist feel | 5.35 | 5.6 | −0.25 |
| Initial overall skin feel | 6.8 | 4.8 | 2 |
| Hour later moist feel | 6 | 5.2 | 0.8 |
| Hour later greasiness/stickiness | 6.9 | 5 | 1.9 |
| Hour later skin smoothness | 5.7 | 5.2 | 0.5 |
| Hour later skin softness | 7.4 | 5 | 2.4 |
| Hour later overall skin feel | 6.8 | 5 | 1.8 |

In addition, when asked to rank the samples as better, worse, or the same, instead of using numbers, the panelists collectively found that the sample according to the present invention (Batch 4) performed as well or better than the comparative sample (Batch 3, hereinafter, Control) in all categories, as shown in TABLE 5 (numbers indicate number of panelists ranking the sample in the category).

TABLE 5

| | Worse than | Same as | Better than |
|---|---|---|---|
| | Control | Control | Control |
| Initial ease of spread | 0 | 0 | 10 |
| Initial adsorption | 0 | 1 | 9 |
| Initial slip | 3 | 5 | 2 |
| Initial tackiness | 0 | 0 | 10 |
| Initial greasiness/stickiness | 0 | 0 | 10 |
| Initial moist feel | 4 | 2 | 4 |
| Initial overall skin feel | 0 | 0 | 10 |
| Hour later moist feel | 1 | 1 | 8 |
| Hour later greasiness/stickiness | 1 | 1 | 8 |
| Hour later skin smoothness | 3 | 3 | 4 |
| Hour later skin softness | 0 | 1 | 9 |
| Hour later overall skin feel | 1 | 0 | 9 |

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A method of improving the aesthetics of a personal care composition which contains a hydrophobic active, comprising:
combining the hydrophobic active with a surfactant and an isocyanate to form an oil phase, and then
reacting the isocyanate with an amine, thereby encapsulating the hydrophobic active in a polyurea shell.

2. The method of claim 1, further comprising emulsifying the oil phase.

3. The method of claim 1, further comprising emulsifying the oil phase with water.

4. The method of claim 1, further comprising emulsifying the oil phase with water and a preservative.

5. The method of claim 3, wherein the amine results from conversion of some isocyanate to amine upon contact with water.

6. The method of claim 1, wherein the amine is a separately added component.

7. The method of claim 1, further comprising first combining the amine with water.

8. The method of claim 3, wherein the step of reacting the isocyanate with an amine includes first combining an amine with water and then adding this mixture to the emulsion incrementally with mixing.

9. The method of claim 1, wherein the hydrophobic active is a moisturizer, emollient, sunscreen, conditioner, vitamin, dye, flavor, or fragrance.

10. The method of claim 1, wherein the hydrophobic active is a sunscreen.

11. The method of claim 1, wherein the hydrophobic active is octyl methoxycinnamate.

12. The method of claim 1, wherein the isocyanate is at least one of diphenylmethane diisocyanate (MDI), Polymeric MDI, polymethylene polyphenyl isocyanate (PAPI), toluene diisocyanate (TDI), isophorone diisocyanate (IPDI), 1,4-phenylene diisocyanate, or hexamethylene diisocyanate (HDI).

13. The method of claim 6, wherein the amine is a multifunctional amine.

14. The method of claim 6, wherein the amine is ethylene diamine.

15. The method of claim 1, wherein the personal care composition demonstrates improved ease of spread and adsorption onto skin.

16. The method of claim 1, wherein the personal care composition demonstrates reduced tackiness and greasiness.

17. The method of claim 1, wherein the personal care composition demonstrates improved ease of spread, improved adsorption onto skin, reduced tackiness, and reduced greasiness.

18. A method of improving the aesthetics of a personal care composition which contains a hydrophobic active, comprising:
combining the hydrophobic active with a surfactant and an isocyanate to form an oil phase,
emulsifying the oil phase with water,
combining a multifunctional amine with water, and
reacting the isocyanate with the amine, thereby encapsulating the hydrophobic active in a polyurea shell.

19. (canceled)

20. A method of improving the aesthetics of a personal care composition which contains a hydrophobic active, comprising
combining the hydrophobic active with a surfactant and a first interfacially polymerizable component to form an oil phase, and then
reacting the first interfacially polymerizable component with a complementary interfacially polymerizable component, thereby encapsulating the hydrophobic active in a polymer shell.
